(19) United States
(12) Patent Application Publication
(10) Pub. No.: US 2011/0124631 A1
(43) Pub. Date: May 26, 2011

(54) TREATMENT OF COGNITIVE DISORDERS WITH CERTAIN ALPHA-7 NICOTINIC ACID RECEPTORS IN COMBINATION WITH ACETYLCHOLINESTERASE INHIBITORS

(76) Inventors: Gerhard Koenig, Newton, MA (US); Dana Hilt, Waltham, MA (US)

(21) Appl. No.: 12/979,139

(22) Filed: Dec. 27, 2010

Related U.S. Application Data
(63) Continuation of application No. 12/777,792, filed on May 11, 2010, now abandoned.
(60) Provisional application No. 61/177,260, filed on May 11, 2009.

(51) Int. Cl. A61K 31/439; A61K 31/473; A61K 31/55; A61P 25/00; A61P 25/28

(52) U.S. Cl. 514/215; 514/305; 514/297

(57) ABSTRACT

A method for improving cognition comprising administering to a patient (R)-7-chloro-N-(quinuclidin-3-yl)benzo[b]thiophene-2-carboxamide or a pharmaceutically acceptable salt thereof and an acetylcholinesterase inhibitor is described together with related compositions.

This application is a Continuation of U.S. application Ser. No. 12/777,792, filed May 11, 2010, which claims priority to U.S. Provisional Application No. 61/177,260, which was filed on May 11, 2009.

### BACKGROUND

Nicotinic acetylcholine receptors (nAChR) form a family of ion channels activated by acetylcholine. Functional receptors contain five subunits and there are numerous receptor subtypes. Studies have shown that central nicotinic acetylcholine receptors are involved in learning and memory. Nicotinic acetylcholine receptors of the alpha7 subtype are prevalent in the hippocampus and cerebral cortex.

WO 03/055878 describes a variety of agonists of the alpha7 nAChR said to be useful for improving cognition. WO 03/055878 suggests that certain agonists of the alpha7 nAChR are useful for improving perception, concentration, learning or memory, especially after cognitive impairments like those occurring for example in situations/diseases/syndromes such as mild cognitive impairment, age-associated learning and memory impairments, age-associated memory loss, Alzheimer's disease (AD), schizophrenia and certain other cognitive disorders. Among the compounds described is (R)-7-chloro-N-(quinuclidin-3-yl)benzo[b]thiophene-2-carboxamide.

Some attribute the cognitive and functional decline observed in Alzheimer's patients to a cholinergic deficient in the central nervous system. At least four drugs that have been used to treat AD, tacrine, donepezil (donepezil HCL; 1-benyzl-4-[(5,6-dimethoxy-1-indanon)-2-yl]methylpiperidine monohydrochloride), rivastigmine ((S)—N-Ethyl-N-methyl-3-[1-(dimethylamino)ethyl]-phenyl carbamate) and galantamine (galantamine hydrobromide; (4aS,6R,8aS)-4a,5,9,10,11,12-hexahydro-3-methoxy-11-methyl-6H-benzofuro[3a,3,2-ef][2]benzazepin-6-ol hydrobromide), appear to act as acetylcholinesterase inhibitors that increase acetylcholine in the CNS.

### SUMMARY

It has surprisingly been found that (R)-7-chloro-N-(quinuclidin-3-yl)benzo[b]thiophene-2-carboxamide can improve cognition in Alzheimer's patients that are being treated with an acetylcholinesterase inhibitor (e.g., donepezil or rivastigmine). The method can improve cognition in patients that have already benefited from an increase in one or more aspects of cognition stemming from the administration of an acetylcholinesterase inhibitor. Thus, a patient already benefiting from acetylcholinesterase inhibitor in one or more aspect of cognition can gain further benefit in one or more aspects of cognition from administration of (R)-7-chloro-N-(quinuclidin-3-yl)benzo[b]thiophene-2-carboxamide and pharmaceutically acceptable salts thereof.

It has also been surprisingly found that memory can be improved when (R)-7-chloro-N-(quinuclidin-3-yl)benzo[b]thiophene-2-carboxamide is administered at a subclinical dose (i.e., a dose that does not improve memory) in combination with an acetylcholinesterase inhibitor that is also administered at a subclinical dose. Thus, a patient can experience a benefit (e.g., improved memory or cognition) from a combination of drugs each of which is administered at very low, side-effect reducing or side-effect avoiding dose. Moreover, the combination of drugs may provide a benefit for a wider range or patients and/or over a longer period of treatment. For example, while certain acetylcholinesterase inhibitors can exhibit reduce efficacy after several months of treatment, the combination may provide a longer period of efficacy.

A patient can benefit in one or more of: speed of processing, attention/vigilance, working memory, visual learning, verbal learning, visual learning, reasoning/problem solving, executive function, and social cognition. Importantly, (R)-7-chloro-N-(quinuclidin-3-yl)benzo[b]thiophene-2-carboxamide and pharmaceutically acceptable salts thereof can be used at low doses in such already treated patients to improve cognition, for example at a daily oral dose of (or no more than): 3 mg, 2.70 mg, 2.50 mg, 2.25 mg, 2 mg, 1.75 mg, 1.50 mg, 1.25 mg, 1 mg, 0.7, 0.5, 0.3 mg or even 0.1 mg. Thus, for example, it can be administered at 0.05-1.5 mg daily dose, preferably 1 mg/daily or 0.3 mg/daily. In the case of administering a dose that is subclinical when administered in the absence of other agents for improving cognition, (R)-7-chloro-N-(quinuclidin-3-yl)benzo[b]thiophene-2-carboxamide and pharmaceutically acceptable salts thereof can be used at a daily oral dose of less than 0.5 mg, 0.3 mg, 0.1 mg, 0.05 mg, 0.03 mg or 0.01 mg. For use at a subclinical level when administered in the absence of other agents for improving cognition, (R)-7-chloro-N-(quinuclidin-3-yl)benzo[b]thiophene-2-carboxamide and pharmaceutically acceptable salts thereof can be used at less than 0.5 nM, 0.4, 0.3, 0.2 or 0.1 nM.

For donepizil, the daily dose used with (R)-7-chloro-N-(quinuclidin-3-yl)benzo[b]thiophene-2-carboxamide or pharmaceutically acceptable salt thereof can be 10 mg, 5 mg, 4.5 mg, 4 mg, 3.5 mg, 3 mg, 2.5 mg, 2 mg, 1 mg or 0.5 mg. The daily dose can be between 5 and 0.5 mg (e.g., 4.5-1.0 mg/day, 4.5-2.0 mg/day, 4.0-2.0 or 2.5 mg/day). For rivistigmine the daily dose for use in combination can be 11, 10, 9, 8, 7, 6 or 5 mg. For galantamine the daily dose for use in combination can be 20, 15, 13, 12, 11, 10, 9, 8, 7, 6 or 5 mg. For acetylcholinesterase inhibitors it is understood that for effectiveness in improving memory or cognition, they must be administered so as to achieve a red blood cell acetylcholinesterase inhibition of at least 65%. In the methods described herein the acetylcholinesterase inhibitor can be administered at a lower dose that achieves only 55% (50, 45, 40, 35 or 30% inhibition).

Described herein is a method for improving cognition comprising administering to a patient (R)-7-chloro-N-(quinuclidin-3-yl)benzo[b]thiophene-2-carboxamide or a pharmaceutically acceptable salt thereof and an acetylcholinesterase inhibitor. In various cases: the patient has been diagnosed with Alzheimer's disease or pre-Alzheimer's disease, the patient has been diagnosed with mild to moderate Alzheimer's disease, the patient has been diagnosed with moderate to severe Alzheimer's disease, the acetylcholinesterase inhibitor is selected from tacrine, donepezil, rivastigmine and galantamine, the acetylcholinesterase inhibitor is selected from donepezil, rivastigmine and galantamine, the acetylcholinesterase inhibitor is selected from donepezil and rivastigmine, the patient has been administered an acetylcholinesterase inhibitor for a period of time prior to being administered (R)-7-chloro-N-(quinuclidin-3-yl)benzo[b]thiophene-2-carboxamide or a pharmaceutically acceptable salt thereof, the prior administration has been for at least one month, the prior administration has been for at least three months, and the prior administration has been for at least six months. In certain cases: the method improves one or more of: learning, delayed memory, attention, working memory, visual learning, speed of processing, vigilance, verbal learning, visual motor function, social cognition, long term memory or executive function.

Also described is a method for improving cognition comprising administering to a patient (R)-7-chloro-N-(quinuclidin-3-yl)benzo[b]thiophene-2-carboxamide or a pharmaceutically acceptable salt thereof at a subclinical dose (a dose that does not improve memory when administered in the absence of an acetylcholinestesterase inhibtor) and an acetylcholinesterase inhibitor, also at a subclinical dose (a dose that does not improve memory when administered in the absence of (R)-7-chloro-N-(quinuclidin-3-yl)benzo[b]thiophene-2-carboxamide or a pharmaceutically acceptable salt thereof). In various cases: (R)-7-chloro-N-(quinuclidin-3-yl)benzo[b]thiophene-2-carboxamide or a pharmaceutically acceptable salt thereof is orally administered at 1.0 mg/day; 0.5 mg/day; 0.3 mg/day; or 0.1 mg/day. In various cases: the acetylcholinesterase inhibitor is donepezil and is orally administered at 5 mg/day; 4.5 mg/day; 4.0 mg/day; 2.5 mg/day; 1.5 mg/day or less; 1.0 mg/day; and the acetylcholinesterase inhibitor is administered at a dose that achieves 10-65% steady state red blood cell acetylcholinesterase inhibition.

Also described is a pharmaceutical composition comprising (R)-7-chloro-N-(quinuclidin-3-yl)benzo[b]thiophene-2-carboxamide or a pharmaceutically acceptable salt thereof and an acetylcholinesterase inhibitor. In various cases: the acetylcholinesterase inhibitor is selected from tacrine, donepezil, rivastigmine and galantamine; the acetylcholinesterase inhibitor is selected from donepezil, rivastigmine and galantamine; the acetylcholinesterase inhibitor is selected from donepezil and rivastigmine; and the acetylcholinesterase inhibitor is donepezil.

Also described is a daily unit dosage pharmaceutical composition comprising no more than 1.0 mg of (R)-7-chloro-N-(quinuclidin-3-yl)benzo[b]thiophene-2-carboxamide or a pharmaceutically acceptable salt thereof, an acetylcholinesterase inhibitor and a pharmaceutically acceptable carrier. In various cases the daily unit dosage pharmaceutical composition comprises no more than 0.5 (0.3, or 0.1) mg of (R)-7-chloro-N-(quinuclidin-3-yl)benzo[b]thiophene-2-carboxamide or a pharmaceutically acceptable salt thereof. In various case the daily unit dosage pharmaceutical composition comprises no more than 5, 4, 3, 2, 1, or 0.5 mg of donepezil.

Also described is a packaged pharmaceuticals comprising a package containing a first unit dosage pharmaceutical composition comprising (R)-7-chloro-N-(quinuclidin-3-yl)benzo[b]thiophene-2-carboxamide or a pharmaceutically acceptable salt thereof and a second unit dosage pharmaceutical composition comprising an acetylcholinesterase inhibitor.

In another aspect, a patient can be treated with an acetylcholinesterase inhibitor of Formula I:

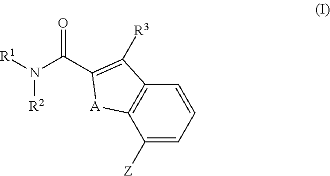

in which

R¹ represents 1-azabicyclo[2.2.2]oct-3-yl,

R² represents hydrogen or C1-C6-alkyl,

R³ represents hydrogen, halogen or C1-C6-alkyl,

A represents oxygen or sulfur, and

Z represents halogen, formyl, carbamoyl, cyano, trifluoromethyl, trifluoromethoxy, nitro, amino, formamido, acetamido, C1-C6-alkyl, C1-C6-alkyoxy, C1-C6-alkylthio, C1-C6-alkylamino, heteroaryl-carbonylamino, arylcarbonylamino, C1-C4-alkylsulfonylamino, di(arylsulfonyl)amino, C3-C6-cycloalkylcarbonylmethyl or amino(hydroxyimino)methyl, or a salt, a solvate or a solvate of a salt thereof in combination with an acetylcholinesterase inhibitor.

In another aspect, the patient is treated with a compound of Formula I wherein R² is hydrogen, R³ is hydrogen, A is sulfur, and Z represents halogen, formyl, carbamoyl, cyano, trifluoromethyl, trifluoromethoxy, nitro, amino, formamido, acetamido, C1-C6-alkyl, C1-C6-alkyoxy, C1-C6-alkylthio, C1-C6-alkylamino, heteroaryl-carbonylamino, arylcarbonylamino, C1-C4-alkylsulfonylamino, di(arylsulfonyl)amino, C3-C6-cycloalkylcarbonylmethyl or amino(hydroxyimino)methyl (particularly halogen, cyano, trifluoromethyl, trifluoromethoxy, methyl, ethyl, methoxy, and ethoxy; more particularly halogen or cyano, even more particularly chloro or cyano).

Described herein are methods for treating a patient by administering a pharmaceutical composition that comprises (R)-7-chloro-N-(quinuclidin-3-yl)benzo[b]thiophene-2-carboxamide or pharmaceutically acceptable salt thereof (e.g., at a daily dose of: 3 mg, 2.70 mg, 2.50 mg, 2.25 mg, 2 mg, 1.75 mg, 1.50 mg, 1.25 mg, 1 mg, 0.7 mg, 0.5 mg, 0.3 mg, 0.1 mg, 0.03 mg or 0.01 mg) in combination with one or more inhibitors of acetylcholinesterase. The treatment can improve one or more facets of cognition (e.g., visual motor skill, learning, delayed memory, attention, working memory, visual learning, speed of processing, vigilance, verbal learning, visual motor function, social cognition, long term memory, executive function, etc.). The patient can have been treated with an acetylcholinesterase inhibitor for a period of time prior to the administration of (R)-7-chloro-N-(quinuclidin-3-yl)benzo[b]thiophene-2-carboxamide. For example, the patient can have been treated with the acetylcholinesterase inhibitor for at least one week, at least one month, at least two months, at least three months, at least four months, at least 6 months or at least one year. The two agents can be administered at the same time either in the same composition or in two different compositions. In addition, the agents can be administered at different times.

Also described herein is a pharmaceutical composition comprising (R)-7-chloro-N-(quinuclidin-3-yl)benzo[b]thiophene-2-carboxamide or a pharmaceutically acceptable salt thereof and a acetylcholinesterase inhibitor (e.g., tacrine, donepezil, rivastigmine or galantamine) and a pharmaceutically acceptable carrier.

“Dose” is the amount of active pharmaceutical ingredient (API) administered to a patient. For example 1 mg means 1 mg of API was orally administered to each patient each day.

“Active Pharmaceutical Ingredient” includes (R)-7-chloro-N-(quinuclidin-3-yl)benzo[b]thiophene-2-carboxamide hydrochloride, (R)-7-chloro-N-(quinuclidin-3-yl)benzo[b]thiophene-2-carboxamide, (R)-7-chloro-N-(quinuclidin-3-yl)benzo[b]thiophene-2-carboxamide hydrochloride monohydrate and (R)-7-chloro-N-(quinuclidin-3-yl)benzo[b]thiophene-2-carboxamide hydrochloride solvate as well as the compounds of Formula I.

Where solvate represents a stoichiometric ratio of 0.1 to 10 molecules of solvent compared to (R)-7-chloro-N-(quinuclidin-3-yl)benzo[b]thiophene-2-carboxamide hydrochloride or (R)-7-chloro-N-(quinuclidin-3-yl)benzo[b]thiophene-2-carboxamide. Solvent molecules include but are not limited too water, methanol, 1,4 dioxane, ethanol, iso-propanol or acetone. In some cases water is the preferred solvate.

### BRIEF DESCRIPTION OF THE DRAWINGS

FIG. 1 depicts the effect of the combination of (R)-7-chloro-N-(quinuclidin-3-yl)benzo[b]thiophene-2-carboxamide (p.o.) and donepezil (p.o.) on cognitive tests in patients.

FIG. 2 depicts the effect of (R)-7-chloro-N-(quinuclidin-3-yl)benzo[b]thiophene-2-carboxamide (p.o.) and donepezil (p.o.) on the discrimination index (d2) in an object recognition task after scopolamine treatment (i.p.) in 5-month-old male Wistar rats (means±SEM). When compared with the vehicle/scopolamine condition, EVP-6124 and donepezil combined reversed the scopolamine-induced memory performance deficit. A difference from the scopolamine condition is depicted with asteriks (Bonferroni t-tests, *: P<0.05). A difference from zero is depicted with # (One sample t-tests, ###: P<0.001).

### DETAILED DESCRIPTION

### (R)-7-chloro-N-(quinuclidin-3-yl)benzo[b]thiophene-2-carboxamide and donepezil or rivastigmine administered to AD patients

The safety and efficacy of (R)-7-chloro-N-(quinuclidin-3-yl)benzo[b]thiophene-2-carboxamide HCl salt was assessed in a Phase 1b study of 48 mild to moderate AD patients 60-80 years of age, on stable donepezil (5 or 10 mg/day) or rivastigmine (6-12 mg/day administered as a twice daily dose, i.e., 3 or 6 mg per dose).

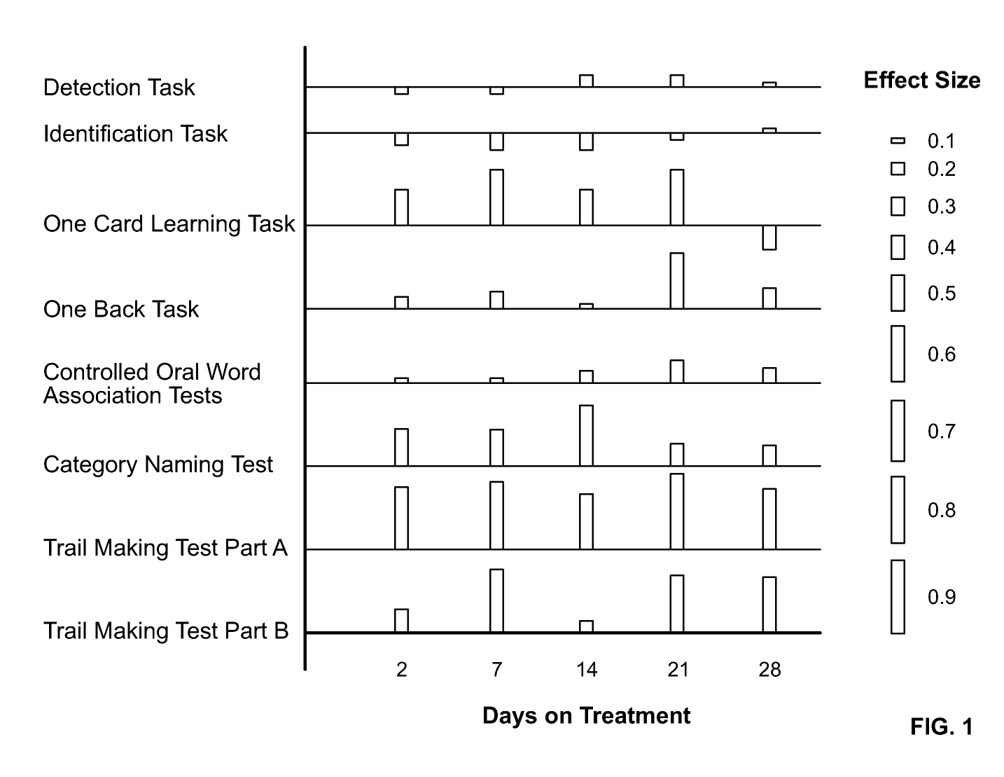

Patients were dosed with placebo or two different doses of (R)-7-chloro-N-(quinuclidin-3-yl)benzo[b]thiophene-2-carboxamide (0.3 or 1.0 mg/d) for 28 days. Safety was evaluated by adverse events, ECG, and clinical laboratory measures. Cognitive effects were measured by CogState computerized cognitive testing and a subset of NTB scales (category fluency, Trails A and B). The results of this analysis are shown in FIG. 1.

(R)-7-chloro-N-(quinuclidin-3-yl)benzo[b]thiophene-2-carboxamide appeared to be safe and well tolerated with no significant adverse events reported more frequently in treated versus placebo patients; there were no SAEs reported. Subjects exposed to (R)-7-chloro-N-(quinuclidin-3-yl)benzo[b]thiophene-2-carboxamide in addition to donepezil or rivastigmine showed an increase in cognitive function, primarily in the domains of non-verbal learning, memory, and executive function.

### (R)-7-chloro-N-(quinuclidin-3-yl)benzo[b]thiophene-2-carboxamide and donepezil at subclinical doses improves memory

The study described below examined the effect of subthreshold doses of the α7 nicotinic receptor agonist (R)-7-chloro-N-(quinuclidin-3-yl)benzo[b]thiophene-2-carboxamide and the AChE inhibitor donepezil on short-term memory deficits, induced by the muscarinic antagonist scopolamine in the object recognition task in 5-months-old male Wistar rats. The object recognition task allows the assessment of consolidation of (object) information into memory (Ennaceur and Delacour, 1988; Prickaerts et al., 1997). In this task a rat is given two trials. In the first trial the rat is put into an arena in which two identical objects are placed. Usually, a rate will inspect the two objects for a certain amount of time. After a certain delay, the rat is given a second trial. In this trial the rat is again placed in the same arena but one of the objects has been replaced by a novel object. Similar to the first trial the rat again explores the two objects. The amount of time exploring each object is recorded in order to determine whether the rat spent a different amount of time exploring the two objects. On basis of this scoring the memory performance can be determined.

Several studies have shown that Wistar rats show a good object memory performance when a one-hour delay is interposed between the first and second trial. However, when a twenty-four hour delay is used the rats do not discriminate between the novel and the familiar object in the second trial, indicating that the rats do not remember the familiar object (i.e. the object presented in both the first trial and the second trial). Using a six-hour delay, the discrimination performance is between the performance of the one hour and twenty-four hour delay, suggesting a delay-dependent forgetting in this task.

A previous study found that 0.3 mg/kg (R)-7-chloro-N-(quinuclidin-3-yl)benzo[b]thiophene-2-carboxamide (p.o.) completely attenuated the object memory deficit induced by the muscarinic antagonist scopolamine (0.1 mg/kg, i.p.), whereas a dose of 0.03 mg/kg had no effect. It was also been found that a dose of 0.3 mg/kg donepezil (p.o.) attenuated the scopolamine-induced object memory deficit, while 0.1 mg/kg donepezil had no effect. In a preliminary study, it was surprisingly found that combined administration of this subthreshold dose of donepezil with a subthreshold dose of (R)-7-chloro-N-(quinuclidin-3-yl)benzo[b]thiophene-2-carboxamide, which neither had an effect on performance when given alone at the subthreshold doses, enhanced memory performance in rats with an object memory deficit induced by scopolamine. This suggests additive synergistic effects between both compounds on cognitive impairment.

In the present study, 30 minutes before the learning trial, rats received an injection with the muscarinic receptor antagonist scopolamine (0.1 mg/kg, administered i.p.). After treatment with scopolamine, rats will show no memory of the objects one hour after the learning trial. It was investigated whether a combination of subthreshold doses of (R)-7-chloro-N-(quinuclidin-3-yl)benzo[b]thiophene-2-carboxamide (0.03 mg/kg, administered p.o.) and donepezil (0.1 mg/kg administered p.o.) could attenuate the scopolamine-induced object memory deficit. All drugs were given 30 minutes before the first trial.

All experimental procedures were approved by the local ethical committee of the Maastricht University for animal experiments and met governmental guidelines. Twenty-four 5-month-old male Wistar rats (Harlan, The Netherlands) were used (average body weights: 465 g). Of these twenty-four animals, only twenty-three animals were included in the final analysis, this was due to the continuous escaping of one the rats. The animals were individually housed in standard type 3 Makrolon cages on sawdust bedding in an air-conditioned room (about 20° C.). They were kept under a 12/12-hour light/dark cycle (lights on from 19.00 to 7.00 h) and had free access to food and water. Rats were housed in the same room as where the animals were tested. A radio, which was playing softly, provided background noise in the room. All testing was done between 9.00 and maximally 18.00 h.

Based on an earlier dose-response study of scopolamine, it was decided that a 0.1 mg/kg dose is the most effective dose to induce memory deficits. Scopolamine hydrobromide was prepared daily and dissolved in saline. Scopolamine was administered i.p. (injection volume 1 ml/kg) 30 min before trial 1. EVP-6124 was dissolved in H₂O. The solution was prepared daily and tested at a dose of 0.03 mg/kg p.o. (injection volume 2 ml/kg). Administration was always 30 min before trial 1 immediately after scopolamine. Donepezil was dissolved in saline. The solution was prepared daily and tested at a dose of 0.1 mg/kg p.o. (injection volume 2 ml/kg). Administration was always 30 min before trial 1 immediately after (R)-7-chloro-N-(quinuclidin-3-yl)benzo[b]thiophene-2-carboxamide.

The vehicle and scopolamine conditions were tested first. Subsequently donepezil, (R)-7-chloro-N-(quinuclidin-3-yl)benzo[b]thiophene-2-carboxamide and the combination of both were tested (n=8 per condition per day of testing). All rats were treated once with each dose condition. But one rat was excluded from the study due to continuous jumping out of the apparatus, thus n=23. The experimentator was unaware of the condition/drugs being tested.

The object recognition test was performed as described elsewhere (Ennaceur and Delacour, 1988). The apparatus consisted of a circular arena, 83 cm in diameter. Half of the 40 cm high wall was made of gray polyvinyl chloride, the other half of transparent polyvinyl chloride. The light intensity was equal in the different parts of the apparatus. Two objects were placed in a symmetrical position about 10 cm away from the gray wall. Each object was available in triplicate. The objects were: 1) a cone consisting of a gray polyvinyl chloride base (maximal diameter 18 cm) with a collar on top made of brass (total height 16 cm), 2) a standard 1 l brown transparent glass bottle (diameter 10 cm, height 22 cm) filled with water, 3) a massive metal cube (10.0×5.0×7.5 cm) with two holes (diameter 1.9 cm), and 4) a massive aluminum cube with a tapering top (13.0×8.0×8.0 cm). A rat could not displace the objects. Fluorescent red tubes and a light bulb provided a constant illumination of about 20 lux on the floor of the apparatus.

A testing session comprised two trials. The duration of each trial was 3 min. During the first trial (T1) the apparatus contained two identical objects (samples). A rat was always placed in the apparatus facing the wall at the middle of the front (transparent) segment. After the first exploration period the rat was put back in its home cage. Subsequently, after a predetermined delay interval, the rat was put back in the apparatus for the second trial (T2), but now with two dissimilar objects, a familiar one (the sample) and a new one. The times spent in exploring each object during T1 and T2 were recorded manually with a personal computer.

Exploration was defined as follows: directing the nose to the object at a distance of no more than 2 cm and/or touching the object with the nose. Sitting on the object was not considered as exploratory behavior. In order to avoid the presence of olfactory trails the objects were always thoroughly cleaned. All combinations and locations of objects were used in a balanced manner to reduce potential biases due to preferences for particular locations or objects.

Several studies have shown that Wistar rats show a good object memory performance when a one-hour delay interposed between the first trial and the second trial. However, when a twenty-four hour delay is used the rats do not discriminate the novel and the familiar in the second trial, indicating that the rats do not remember the object that was presented in the first trial. Using a six hour delay, the discrimination performance is between the performance of the one hour and twenty-four hour delay, suggesting a delay-dependent forgetting in this task. In this study a 1 h interval is used.

In the two weeks, the animals were handled daily and adapted to the procedure in two days, i.e., they were allowed to explore the apparatus (without any objects) twice for 3 min each day. Then the rats were adapted to the testing and i.p. and p.o. injections by a saline injection (1.0 ml/kg and 2.0 ml/kg respectively) 30 min before the first trial until they showed a stable discrimination performance, i.e. a good discrimination at 1-h interval and no discrimination at 24-h interval. Next, the control sessions (vehicle and scopolamine were tested). Subsequently, testing of the drugs (R)-7-chloro-N-(quinuclidin-3-yl)benzo[b]thiophene-2-carboxamide (0.03 mg/kg) and donepezil (0.1 mg/kg) began. Compounds/vehicle were always tested on Monday, Wednesday and Friday (or Tuesday and Thursday) in order to have a sufficient wash-out period between compound sessions.

The basic measures were the times spent by rats in exploring an object during T1 and T2. The time spent in exploring the two identical samples will be represented by ‘a1’ and ‘a2’. The time spent in T2 in exploring the sample and new object will be represented by ‘a’ and ‘1)’, respectively. The following variables were calculated: e1=a1+a2, e2=a+b, and d2=(b−a)/e2 (see Table 1). e1 and e2 are measures of the total exploration time of both objects during T1 and T2 respectively. D2 is a relative measure of discrimination corrected for exploration activity (e2). Thus, there should be no differences in d2 indices between experiments with similar treatments at similar intervals. There were five conditions in this experiment and each rat was subjected to each condition:

1) Vehicle (Scopolamine), Vehicle (Donepezil) & Vehicle ((R)-7-chloro-N-(quinuclidin-3-yl)benzo[b]thiophene-2-carboxamide)
2) Scopolamine, Vehicle (Donepezil) & Vehicle ((R)-7-chloro-N-(quinuclidin-3-yl)benzo[b]thiophene-2-carboxamide)
3) Scopolamine, Donepezil & Vehicle ((R)-7-chloro-N-(quinuclidin-3-yl)benzo[b]thiophene-2-carboxamide)
4) Scopolamine, Vehicle (Donepezil) & (R)-7-chloro-N-(quinuclidin-3-yl)benzo[b]thiophene-2-carboxamide
5) Scopolamine, Donepezil & (R)-7-chloro-N-(quinuclidin-3-yl)benzo[b]thiophene-2-carboxamide

One-sample t-statistics were performed in order to assess per treatment condition whether d2 differed from zero. However, comparison of the value of d2 with the value zero with no variance may not be the most suitable way for analyzing recognition (increased chance of making a type I error). Effects were therefore also assessed by a within subjects (repeated measures) ANOVA. In case of a significant difference between conditions, post hoc analyses with Bonferroni corrections were performed.

TABLE 1
Measures involved in the object recognition test

| Exploration | Discrimination |
|---|---|
| e1 = a1 + a2 | |
| e2 = a + b | d2 = (b − a)/e2 |

e1 is the measure of the time spent in exploring both identical objects (a1 and a2) in the first trial, and
e2 is the measure of the time spent in exploring both the familiar (a) and new object (b) in the second trial;
d2 corresponds to the ability to discriminate between the old and new object during the second trial and is corrected for exploration time during that trial.

The results of the EVP-6124 and donepezil treatment, 30 min before T1, are summarized in Table 2. There were no differences found between treatment conditions in the level of exploration in T1 (e1: F (4, 88)=1.138, n.s.). In T2, there were also no differences between treatment conditions in the level of exploration in T2 (e2: F(4, 88)=0.888, n.s.).

TABLE 2
Results of treatment with the drugs EVP-6124 and donepezil on the measures of object
recognition test after scopolamine treatment

| | | | | | |
|---|---|---|---|---|---|
| IP | vehicle | 0.1 mg/kg scopolamine | 0.1 mg/kg scopolamine | 0.1 mg/kg scopolamine | 0.1 mg/kg scopolamine |
| PO | vehicle | vehicle | 0.1 mg/kg donepezil | vehicle | 0.1 mg/kg donepezil |
| PO | vehicle | vehicle | vehicle | 0.03 mg/kg EVP-6124 | 0.03 mg/kg EVP-6124 |

A) Mean values (± SEM) of total exploration time (s) during the first (e1) and second (e2) trial

| | | | | | |
|---|---|---|---|---|---|
| e1 | 22.74 (2.00) | 18.47 (1.21) | 20.80 (1.75) | 21.08 (1.46) | 20.62 (1.48) |
| e2 | 25.16 (1.95) | 21.65 (1.76) | 21.38 (1.45) | 22.98 (1.67) | 22.02 (2.53) |

B) Mean values (± SEM) of the indices of discrimination (d2) between the new and familiar objects

| | | | | | |
|---|---|---|---|---|---|
| d2 | 0.28 (0.05)*** | 0.02 (0.07) | −0.02 (0.05) | 0.00 (0.05) | 0.34 (0.06)*** |

Scopolamine (i.p.), EVP-6124 (p.o.) and donepezil (p.o.) administration was 30 min before T1.
The delay interval between the first and second trial was one hour.
The d2 measures different from zero are depicted with asteriks (one-sample t-tests, ***: P <0.001).
n = 23 per experimental condition.

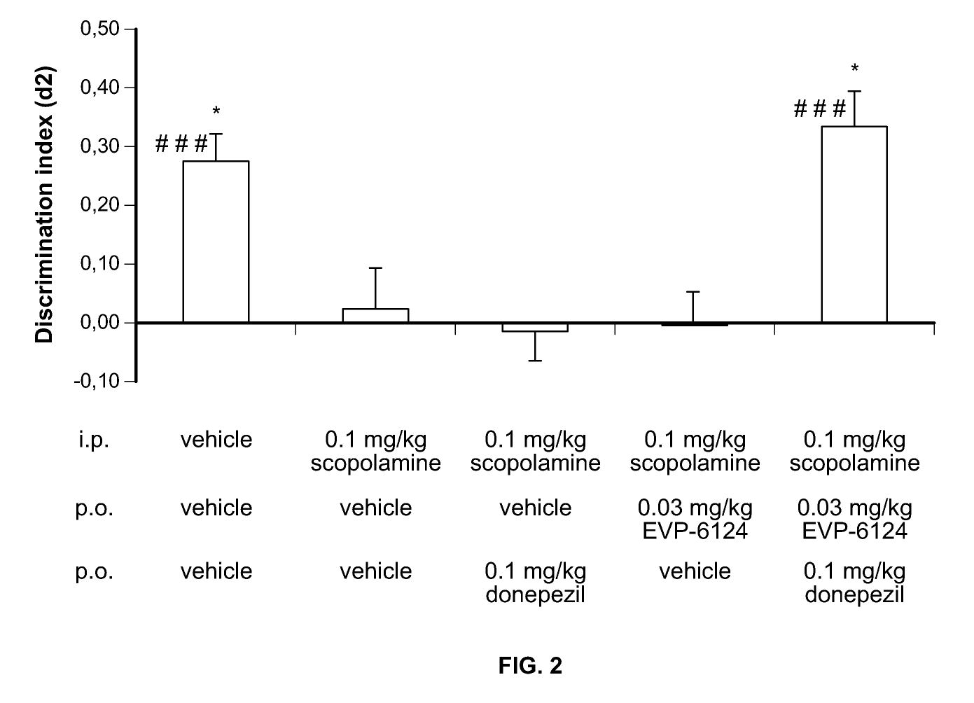

One sample t-tests showed that the d2 value of the combined and vehicle condition differed from zero (see Table 2B). This in contrast to the separately administered scopolamine, (R)-7-chloro-N-(quinuclidin-3-yl)benzo[b]thiophene-2-carboxamide and donepezil conditions, which showed no difference. The effects of (R)-7-chloro-N-(quinuclidin-3-yl)benzo[b]thiophene-2-carboxamide and donepezil treatment on the relative discrimination index d2 are graphically presented in FIG. 1. When comparing between groups, differences were found for the d2 index (F(4, 88)=8.181, P<0.001). The d2 was higher in the combined and vehicle condition than in the scopolamine, (R)-7-chloro-N-(quinuclidin-3-yl)benzo[b]thiophene-2-carboxamide and donepezil conditions (Bonferrone t-tests; see FIG. 2).

For the evaluation of the cognition enhancing effects of combined subthreshold doses of the drugs (R)-7-chloro-N-(quinuclidin-3-yl)benzo[b]thiophene-2-carboxamide (given p.o.) and donepezil (p.o.) after scopolamine administration (i.p.), a 1 h delay interval was used. It was found that in the control vehicle condition the rats remembered the familiar object after such an interval. This is in contrast to the scopolamine (0.1 mg/kg) condition in which no more memory of the familiar object was found. In the present study the dose of scopolamine used for drug testing had no effect on exploratory activity of the animals.

Neither (R)-7-chloro-N-(quinuclidin-3-yl)benzo[b]thiophene-2-carboxamide nor donepezil changed exploratory behavior. The relative discrimination index d2 corrects for alterations in exploratory activity (though these were not observed in the present study). Further it is a reliable measure since the within analysis, i.e. comparison with zero, is able to detect subtle effects on object recognition. The d2 of the donepezil condition as well as that of the (R)-7-chloro-N-(quinuclidin-3-yl)benzo[b]thiophene-2-carboxamide condition were not different from zero. Likewise, between analyses, i.e. comparison with animals treated with scopolamine alone, showed that the subthreshold doses of EVP-6124 (0.03 mg/kg) and donepezil (0.1 mg/kg) did not improve the memory of the animals when given separately. This is in contrast to the combined condition, in which the subthreshold doses of (R)-7-chloro-N-(quinuclidin-3-yl)benzo[b]thiophene-2-carboxamide and donepezil given together completely reversed the object memory dysfunction caused by scopolamine.

### REFERENCES

Boess, F. G., Hendrix, M., van der Staay, F. J., Erb, C., Schreiber, R., van Staveren, W., de Vente, J., Prickaerts, J., Blokland, A., Koenig, G., 2004 Inhibition of phosphodiesterase 2 increases neuronal cGMP, synaptic plasticity and memory performance. Neuropharmacology 47, 1081-1092.
Blokland, A., Prickaerts, J., Honig, W., De Vente, J., 1998. State-dependent impairment in object recognition after hippocampal NOS inhibition. NeuroReport 9, 4205-4208.
Ennaceur, A., Delacour, J., 1988. A new one-trial test for neurobiological studies of memory in rats. 1: Behavioral data. Behay. Brain Res. 31, 47-59.
Ennaceur, A., Meliani, K., 1992. Effects of physostigmine and scopolamine on rats' performances in object-recognition and radial-maze tests. Psychopharmacology 109, 321-330.
Ennaceur, A., Cavoy, A., Costa, J. C., Delacour, J. 1989. A new one-trial test for neurobiological studies of memory in rats. II: Effects of piracetam and pramiracetam. Behay. Brain Res. 33, 197-207.
De Bruin, N. M. W. J., Prickaerts, J., Akkerman, S., Lange, J. H. M., Andriambeloson, E., De Haan, M., Wijnen, J., Van Drimmelen, M., Hissink, E., Heijnk, L. Kruse, C. G. (In Press) SLV330, a cannabinoid CB1 receptor antagonist, ameliorates deficits in the T-maze, object recognition and social recognition tasks in rodents. Neurobiol. Learn. Mem.
Prickaerts, J., Steinbusch, H. W. M., Smits, J. F. M., De Vente, J., 1997. Possible role of nitric oxide-cyclic GMP pathway in object recognition memory: Effects of 7-nitroindazole and zaprinast. Eur. J. Pharmacol. 337, 125-136.
Prickaerts, J., Sik, A., van Staveren, W. C., Koopmans, G., Steinbusch, H. W., van der Staay, F. J., de Vente, J., Blokland, A., 2004. Phosphodiesterase type 5 inhibition improves early memory consolidation of object information. Neurochem Int. 45, 915-928.
Prickaerts, J., Sik, A., van der Staay, F. J., de Vente, J., Blokland, A., 2005. Dissociable effects of acetylcholinesterase inhibitors and phosphodiesterase type 5 inhibitors on object recognition memory: acquisition versus consolidation. Psychopharmacology 177, 381-390.

What is claimed is:

1. A method for improving cognition comprising administering to a patient (R)-7-chloro-N-(quinuclidin-3-yl)benzo[b]thiophene-2-carboxamide or a pharmaceutically acceptable salt thereof and an acetylcholinesterase inhibitor.

2. The method of claim 1 wherein the patient has been diagnosed with Alzheimer's disease or pre-Alzheimer's disease.

3. The method of claim 1 wherein the patient has been diagnosed with mild to moderate Alzheimer's disease.

4. The method of claim 1 wherein the patient has been diagnosed with moderate to severe Alzheimer's disease.

5. The method of any of the forgoing claims wherein the acetylcholinesterase inhibitor is selected from tacrine, donepezil, rivastigmine and galantamine.

6. The method of claim 5 wherein the acetylcholinesterase inhibitor is selected from donepezil, rivastigmine and galantamine.

7. The method of claim 5 wherein the acetylcholinesterase inhibitor is selected from donepezil and rivastigmine.

8. The method of any of the forgoing claims wherein the patient has been administered an acetylcholinesterase inhibitor for a period of time prior to being administered (R)-7-chloro-N-(quinuclidin-3-yl)benzo[b]thiophene-2-carboxamide or a pharmaceutically acceptable salt thereof.

9. The method of claim 8 wherein the prior administration has been for at least one month.

10. The method of claim 9 wherein the prior administration has been for at least three months.

11. The method of claim 10 wherein the prior administration has been for at least six months.

12. The method of any of the forgoing claims wherein the method improves one or more of: learning, delayed memory, attention, working memory, visual learning, speed of processing, vigilance, verbal learning, visual motor function, social cognition, long term memory or executive function.

13. The method of claim 1 wherein one or both of the (R)-7-chloro-N-(quinuclidin-3-yl)benzo[b]thiophene-2-carboxamide or a pharmaceutically acceptable salt thereof and the acetylcholinesterase inhibitor is administered at a subclinical dose.

14. The method of claim 13 wherein (R)-7-chloro-N-(quinuclidin-3-yl)benzo[b]thiophene-2-carboxamide or a pharmaceutically acceptable salt thereof is orally administered at less than 1.0 mg/day.

15. The method of claim 13 wherein (R)-7-chloro-N-(quinuclidin-3-yl)benzo[b]thiophene-2-carboxamide or a pharmaceutically acceptable salt thereof is orally administered at less than 0.5 mg/day.

16. The method of claim 13 wherein (R)-7-chloro-N-(quinuclidin-3-yl)benzo[b]thiophene-2-carboxamide or a pharmaceutically acceptable salt thereof is orally administered at less than 0.3 mg/day.

17. The method of claim 13 wherein (R)-7-chloro-N-(quinuclidin-3-yl)benzo[b]thiophene-2-carboxamide or a pharmaceutically acceptable salt thereof is orally administered at less than 0.1 mg/day.

18. The method of claim 13 wherein the acetylcholinesterase inhibitor is donepezil and is orally administered at less than 5 mg/day.

19. The method of claim 13 wherein the acetylcholinesterase inhibitor is donepezil and is orally administered 4.5 mg/day or less.

20. The method of claim 13 wherein the acetylcholinesterase inhibitor is donepezil and is orally administered at 4.0 mg/day or less.

21. The method of claim 13 wherein the acetylcholinesterase inhibitor is donepezil and is orally administered at 2.5 mg/day or less.

22. The method of claim 13 wherein the acetylcholinesterase inhibitor is donepezil and is orally administered at 1.5 mg/day or less.

23. The method of claim 13 wherein the acetylcholinesterase inhibitor is donepezil and is orally administered at than 1.0 mg/day or less.

24. The method of claim 1 wherein the acetylcholinesterase inhibitor is administered at a dose that achieves 10-65% steady state red blood cell acetylcholinesterase inhibition.

25. A pharmaceutical composition comprising (R)-7-chloro-N-(quinuclidin-3-yl)benzo[b]thiophene-2-carboxamide or a pharmaceutically acceptable salt thereof and an acetylcholinesterase inhibitor.

26. The pharmaceutical composition of claim 25 wherein the acetylcholinesterase inhibitor is selected from tacrine, donepezil, rivastigmine and galantamine.

27. The pharmaceutical composition of claim 25 wherein the acetylcholinesterase inhibitor is selected from donepezil, rivastigmine and galantamine.

28. The pharmaceutical composition of claim 25 wherein the acetylcholinesterase inhibitor is selected from donepezil and rivastigmine.

29. The pharmaceutical composition of claim 25 wherein the acetylcholinesterase inhibitor is donepezil.

30. A daily unit dosage pharmaceutical composition comprising no more than 1.0 mg of (R)-7-chloro-N-(quinuclidin-3-yl)benzo[b]thiophene-2-carboxamide or a pharmaceutically acceptable salt thereof, an acetylcholinesterase inhibitor and a pharmaceutically acceptable carrier.

31. The daily unit dosage pharmaceutical composition of claim 30 comprising no more than 0.5 mg of (R)-7-chloro-N-(quinuclidin-3-yl)benzo[b]thiophene-2-carboxamide or a pharmaceutically acceptable salt thereof.

32. The daily unit dosage pharmaceutical composition of claim 31 comprising no more than 0.3 mg of (R)-7-chloro-N-(quinuclidin-3-yl)benzo[b]thiophene-2-carboxamide or a pharmaceutically acceptable salt thereof.

33. The daily unit dosage pharmaceutical composition of claim 31 comprising no more than 0.1 mg of (R)-7-chloro-N-(quinuclidin-3-yl)benzo[b]thiophene-2-carboxamide or a pharmaceutically acceptable salt thereof.

34. The daily unit dosage pharmaceutical composition of claim 31 comprising no more than 5 mg of donepezil.

35. The daily unit dosage pharmaceutical composition of claim 31 comprising no more than 4 mg of donepezil.

36. The daily unit dosage pharmaceutical composition of claim 31 comprising no more than 2.5 mg of donepezil.

37. The daily unit dosage pharmaceutical composition of claim 31 comprising no more than 1 mg of donepezil.

38. Packaged pharmaceuticals comprising a package containing a first unit dosage pharmaceutical composition comprising (R)-7-chloro-N-(quinuclidin-3-yl)benzo[b]thiophene-2-carboxamide or a pharmaceutically acceptable salt thereof and a second unit dosage pharmaceutical composition comprising an acetylcholinesterase inhibitor.
